(19) United States
(12) Patent Application Publication
(10) Pub. No.: US 2013/0035604 A1
(43) Pub. Date: Feb. 7, 2013

(54) Frequency Analysis of 12-Lead Cardiac Electrical Signals to Detect and Identify Cardiac Abnormalities

(71) Applicant: Cecilia Yu, Hacienda Heights, CA (US)

(72) Inventor: Cecilia Yu, Hacienda Heights, CA (US)

(21) Appl. No.: 13/195,797

(22) Filed: Aug. 1, 2011

(51) Int. Cl. A61B 5/0402

(52) U.S. Cl. 600/509

(57) ABSTRACT

A method to detect and identify any cardiac abnormality of a human heart by means of the frequency analysis of the cardiac electrical signal of the twelve (12) leads independently and two (2) corresponding leads jointly, comprises the steps of obtaining from a patient 12 time-domain cardiac electrical signals, commonly known as 12-lead ECG (electrocardiogram) signals, mathematically transforming these ECG signals into twelve (12) individual frequency-domain amplitude spectra with one spectrum for each of the 12 leads in a frequency range from 0 Hz to 25 Hz, applying the digital signal process principles of plurality of functions to determine the quality and quantity of each signal and that of two corresponding signals, comparing against a set of parameters that has been established in advance to identify and determine the diagnostic value of each index, and analyzing the value of all identified indexes thereby assessing the pathological condition of a human heart.

### FIELD OF INVENTION

The present invention is in the field of cardiology and is a method whereby a set of time-domain cardio electrical signals, commonly known as ECG (electrocardiogram) signals, is collected by a plurality of detecting electrodes and mathematically transformed into frequency-domain spectra in a low frequency range of 0 to 25 Hz. By applying the digital signal process principles, the present invention provides a method to study the quantity and quality of those signals to obtain a wide range of vital information relating to the pathological condition of a human heart. From this vital information, it can detect and identify cardiac abnormality in a human heart.

### DESCRIPTION OF THE PRIOR ART

Heart diseases have been the leading cause of death in the United States and a major concern in the medical field over the years. With the invention of electrocardiogram (ECG) technology more than 100 years ago, physicians have been interpreting the changes in the ECG to detect various heart diseases such as dysrhythmias, heart size and position, conduction system, and cardiac ischemia or infarction. The advantage of interpreting ECG is this technique is non-invasive, but the major drawback is that it provides less than 50% in accuracy with even less in specificity. In the last twenty years, with the advances in microprocessors, ECG interpretation has been computerized to eliminate the human error, however since the changes in ECG are generally very minor and in some case none, the improvement in accuracy and specificity has been rather limited. There are many other technologies available to the doctors for the detection of heart diseases, such as the nuclear scanning which is non-invasive but expensive to run, catheterization or coronary angiography which is an invasive and expensive procedure. These testing procedures have often been used as a last test to confirm the existence of heart diseases after positive finding in the preliminary testing.

From the technical point of view, an ECG is a compilation and recording of a number of different and complex cardiac electrical signals in a time sequence. When an area of heart muscle is damaged due to lack of blood supply, inflammation or any other reasons, the characteristic of electrical currents traveling through the damaged heart muscle is affected with changes in amplitude and/or direction. Some of the changes can be detected by observation of changes in ECG and a proper diagnosis can be made. But often time the changes are so subtle or minor that they are undetectable by examining an ECG.

In 1965, Fast Fourier Transformation (FFT), a very efficient algorithm, was developed to implement the Discrete Fourier Transformation. With the invention and advance of computer technology, Fast Fourier Transformation can transform a complex ECG time-domain signal into its unique frequency components in a few seconds. In the recent years, a tremendous amount of research work has been done using FFT to analyze the ECG to detect heart disease. For instance, Chamoun's patent (U.S. Pat. No. 5,020,540) described a method and system of choosing and extracting an arrhythmia-free QRST complex from a time-domain ECG as a template and analyzing its frequency components in a very high frequency range (150-250 Hz) to detect various types of heart diseases. The shortcomings in this approach are two-fold, one is that Chamoun's per-determination to use only an arrhythmia-free QRST complex for frequency analysis which artificially excludes a group of patients from testing. The second shortcoming is that Chamoun's patent only uses the high frequency components in the range of 150 to 250 Hz for the analysis when a major portion of the cardiac electrical frequency components after FFT transformation are in the 0 to 50 Hz frequency range. Chamoun's patent of the low frequency components from 0 Hz to 25 Hz of an ECG complex leaves a big gap in the research spectrum. The present invention without predetermination of which segment of ECG signal should be use looks at the entire cardiac electrical signals in their low frequency range of 0 to 25 Hz where a treasury of useful information is located.

At the time of Chomoun's patent, Shen's patent (U.S. Pat. No. 5,029,082) revealed an apparatus using 12-lead electrocardiography (ECG), 3-lead vectorcardiography (VCG) and 2-lead frequency-domain analysis for the diagnosis of heart diseases and evaluation of health. In the 2-lead frequency-domain analysis, Shen's patent only analyzed the cardiac electrical signals from two leads, namely lead V5 and lead II and thus overlooked any vital information from other ten leads. Later, Feng in his patents (U. S. Pat. No. 5,509,425, No. 5,542,429, and No. 5,649,544) carried out more research work in frequency single analysis using the same two lead ECG signals, lead II and lead V5, the same leads used in Shen's patent. All three of Feng's patents describe a method to mathematically determine a plurality of functions and a set of indices for each function for diagnosing a cardiac condition and warning of heart attack of a patient. However, there are the same shortcomings in Feng's approach as in Shen's. Both Shen's and Feng's patents only analyzed the ECG signals collected from two selected leads, II and V5, for their frequency analysis. They both failed to give due consideration of the useful information the cardiac electrical signals form other ten ECG leads may have and thus unnecessarily forfeited the benefit from their analysis.

After Feng's patents, frequency analysis of ECG signals from all 12 leads was described later in Fang's patents (U.S. Pat. No. 6,148,228, No. 6,638,232 B1 and No. 6,936,010 B2). All three patented are entitled “System and Method for Detecting and Locating Heart Diseases.”, and U.S. Pat. No. 6,638,232 B1 and No. 6,936,010 B2 are continuation of patent application Ser. No. 09/035,476, filed on Mar. 5, 1998, now U.S. Pat. No. 6,148,228. Fang's patents analyze 12 cardiac electric signals in frequency domain, and establish a base value by multiplying a patient's heart beats per second by a scaling quantity of 5, and then comparing the area of a power spectrum from 0 Hz to the base value over the area from said base value to infinite to get an area ratio, and then using the area ratio to establish an evaluation standard indicative of coronary artery diseases. Furthermore Fang's patents provide a means to conduct peak analysis of the power spectrum, and a scheme for locating detected heart disease. The shortcomings in Fang's patents are that they analyzed the individual cardiac electric signals with the application of only one of the multiply functions of the digital signal process, namely the power spectrum. They fail to provide means to study the relationship between two or more leads and thus forfeit a wealth of the vital and valuable information that can be obtained from analyzing the performance of two or more inter-related lead such as phase shift, impulse response, correlation or coherence, functions commonly used in digital signal process.

The present invention provides a method for a systematic approach to analyze in frequency domain the quality and quantity not only each lead independently but also the relationship between two correspondent leads. It cures the deficiencies from both Feng's and Fang's patented inventions.

### SUMMARY OF THE INVENTION

An object of the present invention is to provide a method to analyze the 12-lead cardiac electrical signals in low frequency range, 0 to 25 Hz, to detect and identify abnormalities for pathological evaluation of a human heart.

Another object of the present invention is to provide a method for synchronously correlatively analyzing all twelve cardiac electric signals.

Another object of the present invention is provide a method to apply multiple functions of digital signal processes to analyze multiple cardiac electrical signals

Another object of the present invention is to provide a method of scoring various diagnostic indexes to assess a normal or abnormal pathological condition of a heart.

Yet another object of the present invention is to provide a method of compiling and comparing various indexes to determine the synchronization, correlation and coherence between different traveling cardiac electrical currents to detect and identify cardiac abnormality.

### BRIEF DESCRIPTION OF THE PRESENT INVENTION

The present invention describes a method for assessing the pathological condition of a human heart by first collecting and digitizing the ECG signals, applying multiple functions of the digital signals processing to calculate a plurality of diagnostic parameters, and comparing the calculated parameters against those established in advance to assess the overall pathological condition of a patient's heart.

It has been long established by the medical professions that each lead of the 12-lead in ECG looks at a certain area of the heart. For the six chest leads, lead-aVL looks at the left atrium and lateral of the left ventricle; lead-I looks at the lateral wall of the left ventricle; lead-aVR looks at the right atrium and upper portion of the right ventricle, from its perspective on the right shoulder; lead-II looks at the inferior wall of the left ventricle; lead-aVF looks at the inferior wall of the left ventricle; and lead-III looks at the inferior wall of the left ventricle. For the six limb leads, lead-V1 looks at the right ventricle and septum; lead-V2 looks at the right ventricle and septum; lead-V3 looks at the anterior wall of the left ventricle; lead-V4 looks at the anterior wall of the left ventricle; lead-V5 looks at the anterior and lateral wall of the left ventricle; and lead-V6 looks at the lateral wall of the left ventricle. To sum up, there are two or more leads that look at different areas of the heart: leads V1 and V2 look at the septal wall; leads V3 and V4 look at the anterior wall of the left ventricle; leads V5 and B6 look at the anterior and lateral wall of the left ventricle; Leads II, III and aVF look at the inferior wall of the left ventricle; leads V1 to V6 together look at the overall anterior wall of the left ventricle; and lead aVR looks at the endocardial wall to the surface of the right atrium.

The improvement provided by this present invention over other patented inventions lies in its capability to provide a method to examine the characteristic of cardiac electric current detected by each lead in the power spectrum in addition to the relationship between two or more leads to provide an assessment of the pathological condition of a human heart as a whole. It also provides a simple, fast, easy-operation and non-invasive procedure, just like how ECG is done over 100 years, but with an much improved accuracy rate in the preliminary test for heart diseases comparing to the average of 50% by a conventional rest ECG.

### BRIEF DESCRIPTION OF THE DRAWINGS

FIG. 1 is a block diagram of the apparatus components used in the present invention;

FIG. 2 is a signal processing flow chart showing the steps from the collection to processing of the cardiac electric signals;

FIG. 3 is a graphical depiction of a 12-lead electrocardiogram (ECG);

FIG. 4 is a graphical depiction of twelve spectra with one for each lead;

FIG. 5 is a picture showing five different digital signal processing functional frequency spectra with one for each function.

FIG. 6 is a flow chart showing the multi-functional correlative analysis descried in the embodiment of the present invention;

FIG. 7 is the graphical depiction of one of the diagnostic assessment of the pathological condition of a human heart showing where the disease lies.

FIG. 8 is a table for all the diagnostic indexes and their respective positive or negative score.

### DESCRIPTION OF THE PREFERRED EMBODIMENTS

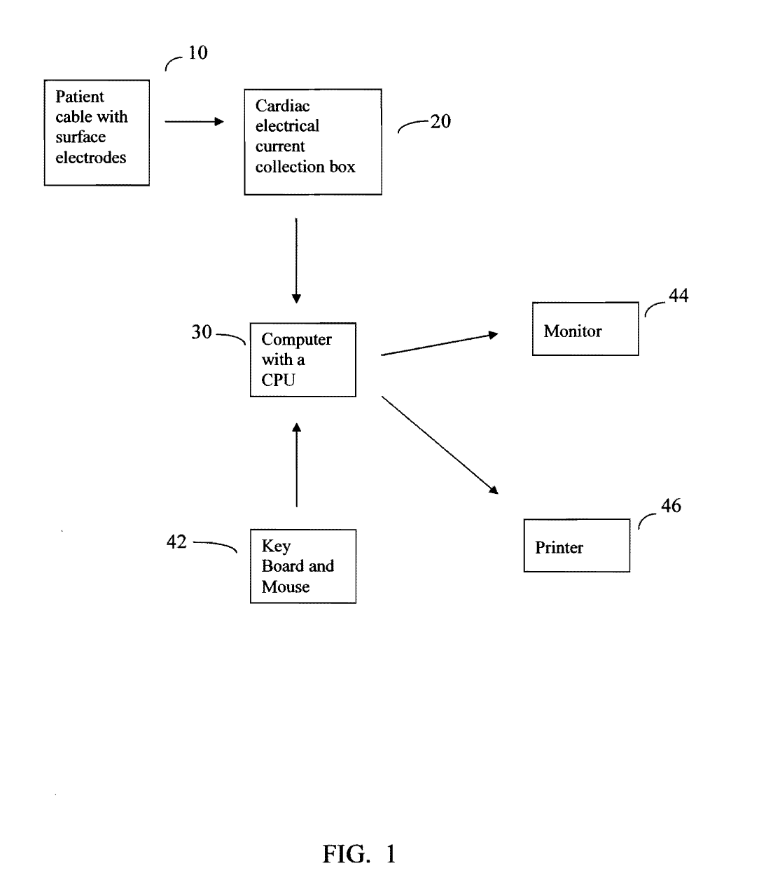

Referring to FIG. 1, the apparatus used in the present invention consists of a conventional 12 lead electrocardiograph (ECG) patient cable with the surface electrodes 10. A data collection box 20 to collect the cardiac electrical signals from a patient. The data collection box 20 has two connecting ports: a patient cable port 22 and a USB port 24, and the function of the data collection box is to collect, filter and sort the cardiac electrical signals to rid of an incidental electrical activity or artifact that may cause interference to the cardiac signal, and to amplify, digitize and convert the time-domain ECG signal to its frequency components.

One end of the patient cable 10 is connected to the body surface of a patient via electrodes at ten prescribed positions with the other end with a patient cable connector connected to a patient cable port of a data collection box 20. After the patient cable 10 is successfully connected to a patient and data collection box 20, the data collection box 20 is then connected through its USB port to a computer 30. This computer 30 has a CPU unit with a proprietary diagnostic analysis software already installed to process (filter, sort, amplify and digitize) the information and mathematically calculate and assemble the data. The software also contains a set of proprietary per-selected diagnostic indexes and their score for comparative diagnostic purpose. For operation, the computer 30 can be connected to a keyboard and mouse 42, a monitor 44 and a printer 46. The monitor 44 provides visual displays and the printer 46 is to print out the output information on command.

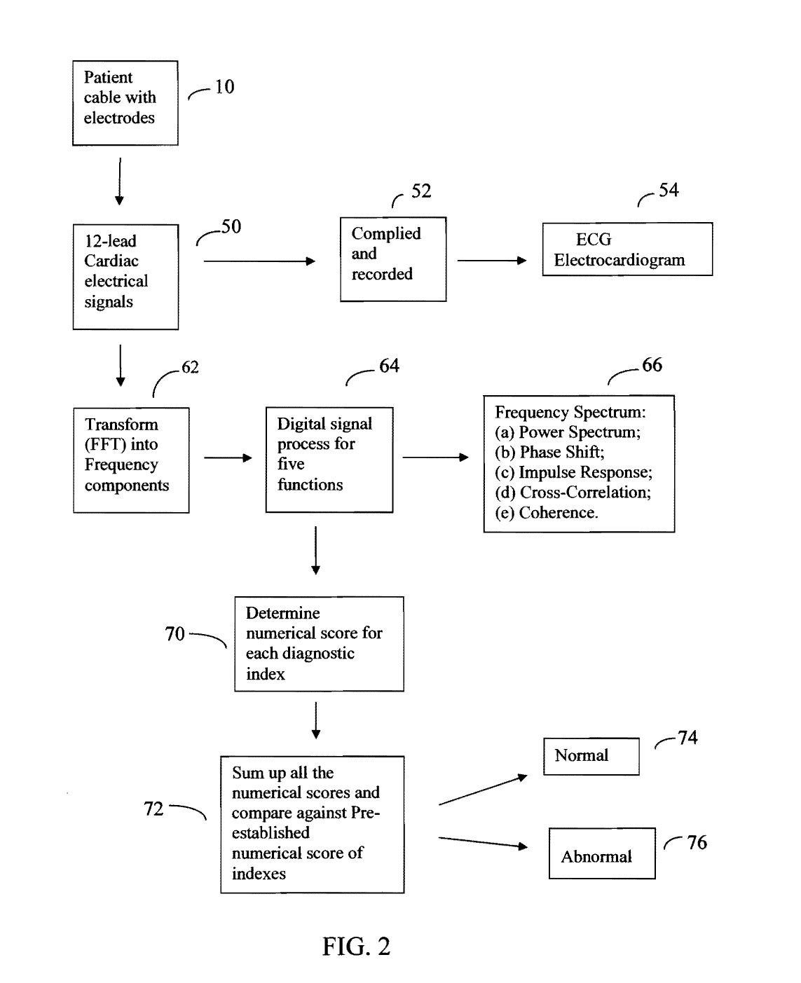

Referring to FIG. 2, it is a signal processing flow chart showing how a cardiac electrical signal is processed by the method described in the present invention. The time-domain cardiac electrical signals 50 collected by the patient cable 10 of a tested subject are first compiled and recorded 52 and displayed as Electrocardiogram (ECG) 54. Concurrently, the same signals are mathematically transformed by means of Fast Fourier Transformation (FFT) into their frequency components 62. These frequency data 62 are digitally analyzed through mathematically calculations 64 and resulting spectrum for each of five different DSP (Digital Signal Process) functions were presented 66. These five are Power Spectrum 66(a), Phase Shift Spectrum 66(b), Impulse Response Spectrum 66(c), Cross-correlation Spectrum 66(d), and Coherence Spectrum 66(e). After calculation of five functions, a set of diagnostic indexes and their respective score is then established 66(f). The pathological diagnosis is done by comparing this set of data 66(f) against a pre-selected set of diagnostic indexes and their score 68 that has already been installed in the computer 30. A detailed index table is then developed and presented as being shown in FIG. 8 listing all the indexes against twelve ECG leads.

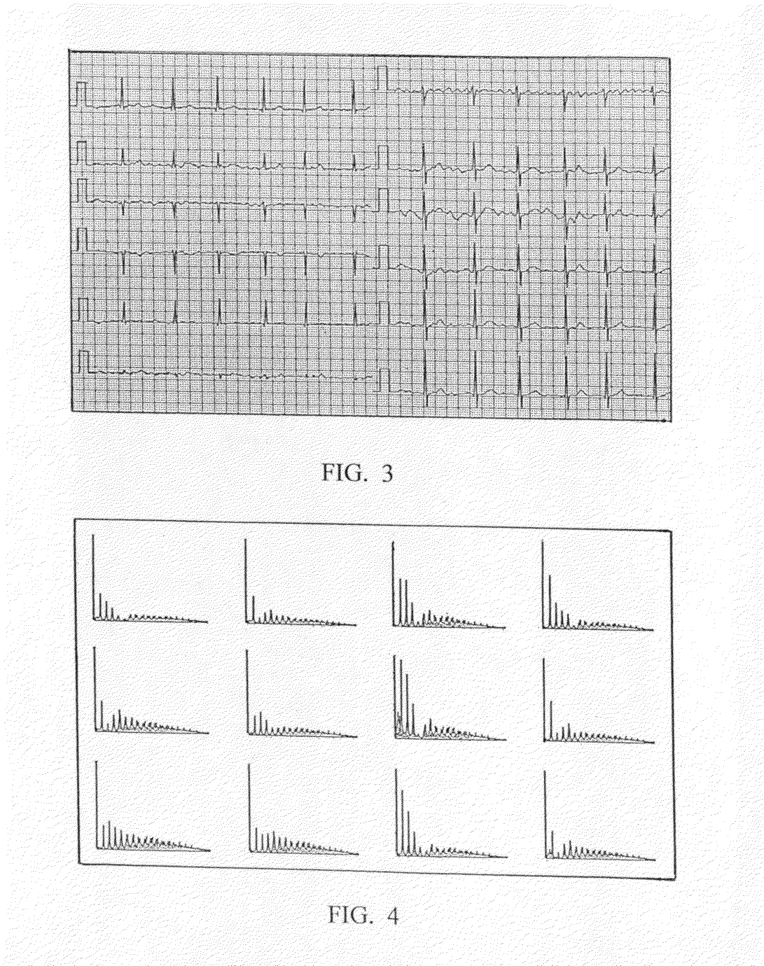

FIG. 3 is a graphical depiction of an electrocardiogram (ECG) 54. The ECG shows 12 electrocardiograms with one for each lead. For the limb leads: I, II, III, aVR, aVL, aVF; and for the chest leads: V1, V2, V3, V4, V5, and V6.

As shown in FIG. 2, the time domain cardiac electrical currents 50 will be transformed in their frequency components 62. Those components will then be mathematically calculated for five digital signal process functions 64. The calculation results are shown in five different forms of frequency spectrum 66. A collection of 12 lead power spectra from one patient with one spectrum for each lead is shown in FIG. 4. There are six for the limb leads and six for the chest leads.

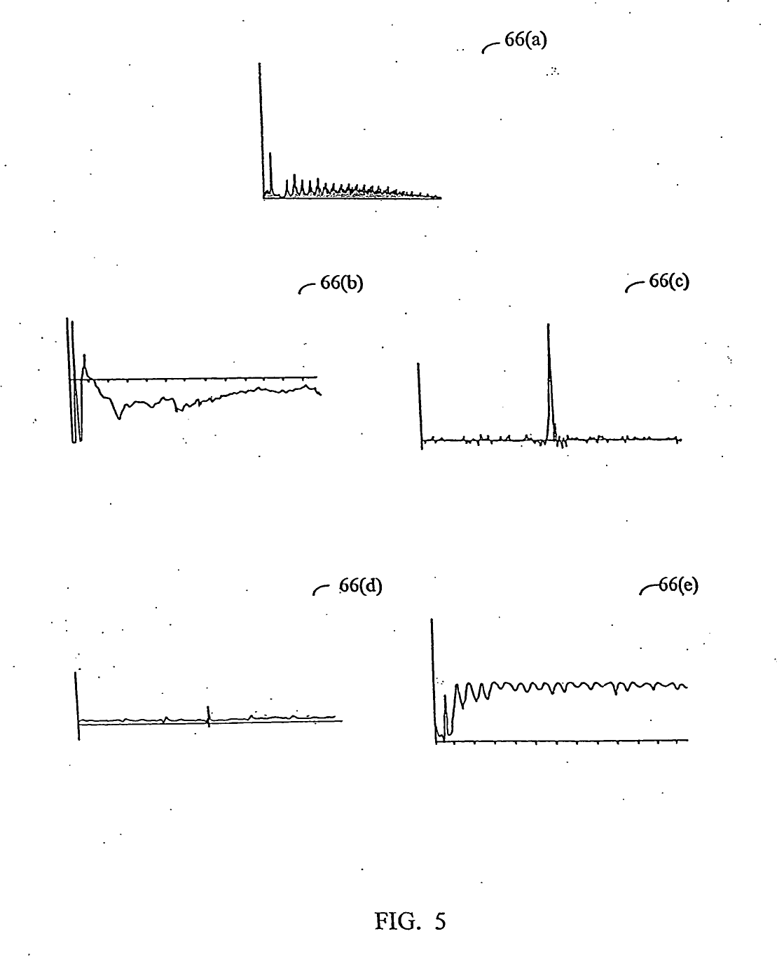

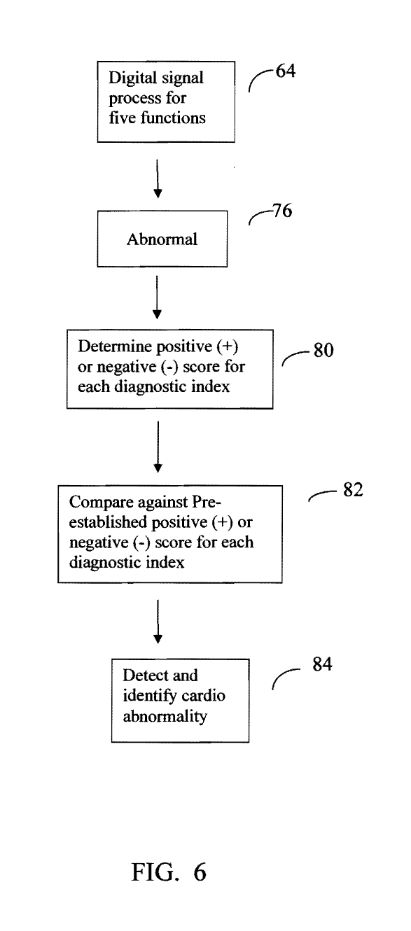

FIG. 5 is a graphical depiction showing five different digital signal processing functional frequency spectra: 66(a) is a power spectrum of a single lead and it is used to examine the behavior of a single signal at every moment in time; 66(b) is a phase shift spectrum showing whether the two cardiac electrical currents from two different parts of the heart travel in phase or whether there is a time lag between the two; 66(c) is an impulse response spectrum showing whether the electrical current detected from two different leads are mirror image of each other or not; 66(d) is a cross-correlation spectrum showing any degree of mutual match of the electrical activities from different parts of the heart; and 66(e) is a coherence spectrum showing the degree of coherence in amplitude, frequency and phase angle of two different cardiac electrical signals from two different parts of the heart.

To diagnose abnormalities, the method of the present invention consists of two steps of the diagnostic evaluation process. The first step is illustrated in the FIG. 2. It consists of five mathematically calculations of the frequency data 62 using five functional equations in digital signal processing 64. From the calculated results of each of the five digital signal process functions, a group of indexes is identified and a diagnostic score is determined and assigned to each index 70. For example, if the main peak of the phase shift spectrum is upside down, an index ‘PV” is assigned to this observation and a score of “5” is given to this index. All together there are 31 such scoring indexes have been selected and each index is given a numerical score from 0 to 5. After all the indexes have been identified and scored, all individual scores are added up to determine a total scoring sum 72. For a scoring below 25, the heart is deemed “Basically Normal” 74 and no further diagnostic analysis will be done. However, for a score that is equal to or over 25, the heart is deemed “Abnormal” 76. When the diagnosis results in “Abnormal”, the method in the present invention will go to the second step for further diagnostic analysis 80.

The second step of diagnostic analysis 80 of the present invention is a continuous analysis of the information from the mathematically calculation of the frequency data 62 using the five functional equations in digital signal processing 64. More diagnostic indexes are utilized to give a wider evaluation of the pathological condition of a heart. In the first step of the present invention, thirty-one indexes are used, but in the second step of analysis 80, the index number is increased to fifty-three which includes some but not all the indexes used in the step one 70.

The second step 80 also utilizes a scoring system with positive (+) and negative (−) scoring, not the numerical scoring as in step one 70. After the calculation 62 and 64, each index is given a positive (+) or negative (−) score 82. Comparing all the positive (+) indexes established in 82 against a pre-established diagnostic table 68 that has already been stored in the computer 30, the present invention will from the positive (+) or negative (−) to identify any existence of heart diseases such as dysrhythmias, electrical conduction block or cardiac ischemia or infarction. By identifying the presence of one or more likely heart diseases, the method in the present invention thereby provides the underlying causes for the Abnormality diagnostic evaluation.

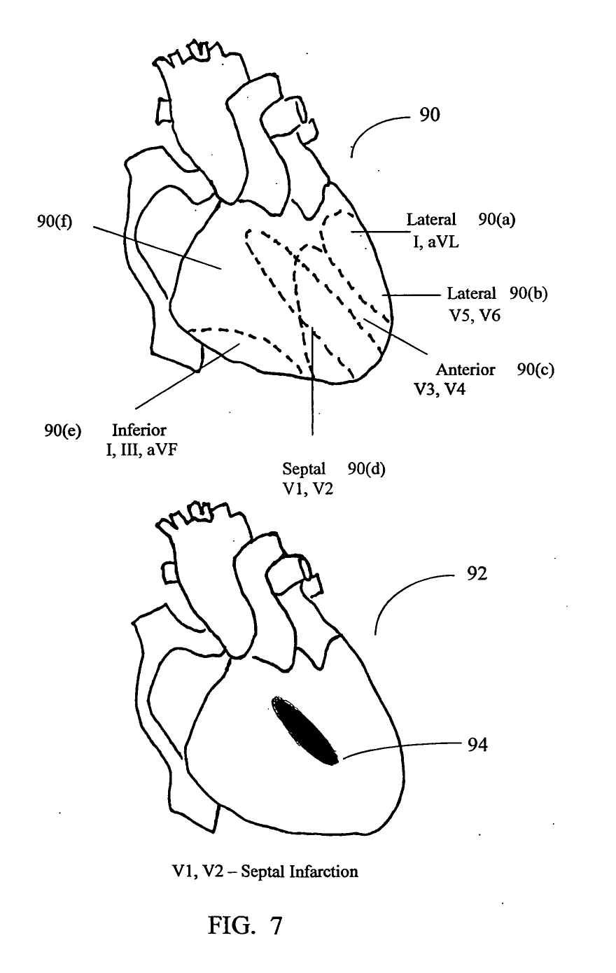

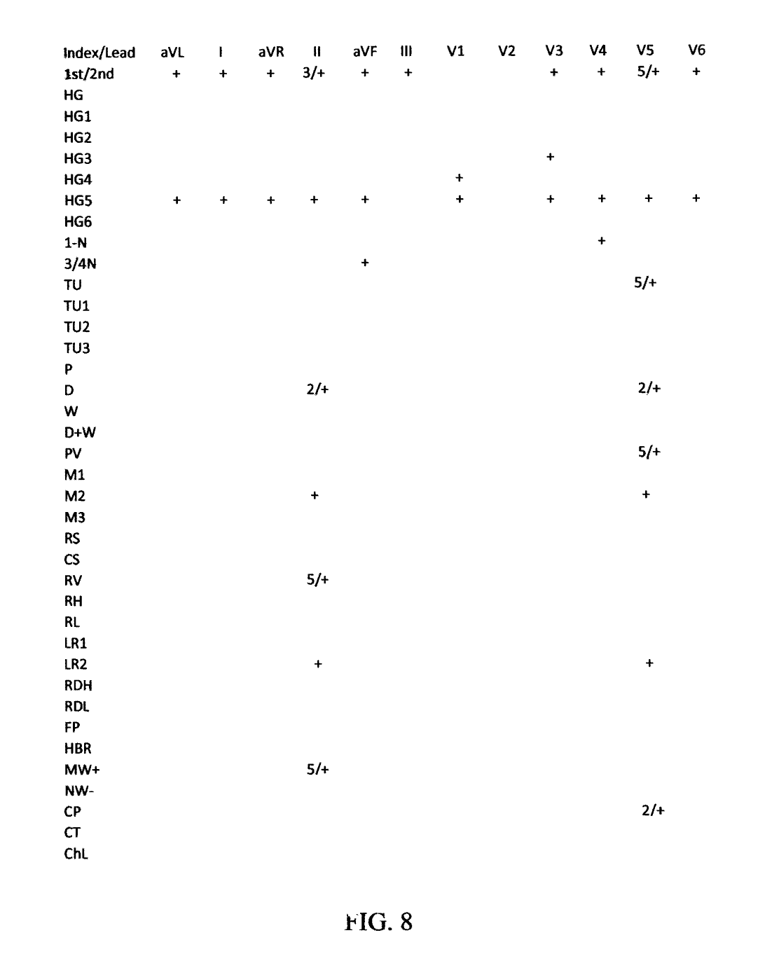

Referring to FIG. 7, a graphical depiction showing one of the diagnostic assessment of the pathological condition of a human heart from step two 80 of the present invention. It consists of two graphic drawings of a human heart. The one on the top of the page of FIG. 7 depicts a heart with several areas circled and lines with area identified and ECG leads pointing to each circle 90. In detail, 90(a) is the upper lateral wall of the left ventricle where the leads I and aVL look at; 90(b) is the lower lateral wall of the left ventricle where the leads V5 and V6 look at; 90(c) is the anterior wall of the left ventricle where the leads V3 and V4 look at; 90(d) is the septal wall of the heart where the leads V1 and V2 look at; 90(e) is the inferior wall of the left ventricle where the leads II, III, and aVF look at; and 90(f) is the right atrium the lead aVR looks at. The second picture 92 in FIG. 7 depicts a heart with an area darken 94. This darken area in 94 indicates where some heart disease has been identified. Right below the picture 92, there is a diagnostic result 96 stating what type of heart disease has been detected and identified. In this example, the diagnosis has found the patient may have “Septal Infarction identified by abnormalities in the area where the cardiac electrical current is detected by the leads V1 and V2 of the ECG.”

What is claimed is:

1. A method for non-invasively evaluating the condition of heart comprising the steps of:
obtaining time-domain cardiac electrical signals from a patient using a conventional electrocardiograph (ECG) patient cable with ten surface electrodes;
mathematically transforming the time-domain cardiac electrical signals into their frequency-domain components;
determining the performance of a plurality of digital signal processing functions from the frequency-domain components;
generating a number of diagnostic indexes for each function;
comparing said diagnostic indexes to pre-selected diagnostic indexes to assign a numerical score for each of said diagnostic indexes of said patient; and
assessing said pathological condition of said patient's heart from said sum of said score from all diagnostic indexes.

2. The method of claim 1 wherein said time-domain cardiac electrical signals are signals from all 12 leads.

3. The method of claim 1 wherein said mathematically transforming time-domain cardiac electrical signals into frequency-domain components uses Fast Fourier Transformation equations;

4. The method of claim 1 wherein the transformation from time-domain signals into frequency domain components is done concurrently for all 12 leads.

5. The method of claim 1 wherein said frequency-domain components are frequency components in a low frequency range from 0 Hz to 25 Hz.

6. The method of claim 1 wherein said plurality of digital signal processing functions consists of means to calculate the power spectrum, phase shift, impulse response, cross-correlation, and coherence;

7. The method of claims 1 wherein said number of diagnostic index generated for each function is 1 to 50;

8. The method of claim 1 wherein said numerical score for each of said diagnostic indexes for said patient is 0 to 10.

9. The method of claim 1 wherein said sum of numerical score from said diagnostic indexes for assessing pathological condition of said patient's heart is in a range from 1 to 100.

10. The method of claim 1 further comprising steps of
measuring diagnostic value of said each index;
scoring each index a positive (+) index or a negative (−) index after comparing said diagnostic value of said index to said pre-established diagnostic value of said pre-selected index.
comparing all said positive (+) indexes to reference per-selected indexes; and
detecting presence of heart disease.

11. The method of claim 9 wherein number of said indexes is 1-100.

12. The method of claim 9 wherein each index is identified by alphabetic letters.

13. The method of claim 9 wherein a positive (+) index indicates an abnormal condition and a negative (−) index indicates a normal condition.

14. The method of claim 9 wherein said detecting presence of heart disease is done by positive (+) index comparison against a set of pre-established indexes.
